(19) United States
(12) Patent Application Publication
(10) Pub. No.: US 2005/0004037 A1
(43) Pub. Date: Jan. 6, 2005

(54) Osteogenic growth oligopeptides as stimulants of hematopoiesis

(75) Inventors: Itai Bab, Carmei Yosef (IL); Michael Chorev, Jerusalem (IL); Arye Shteyer, Mevasseret Zion (IL); Andras Muhlrad, Jerusalem (IL); Nura Mansur, Ramla (IL); Olga Gurevitch, Jerusalem (IL); Zvi Greenberg, Jerusalem (IL); Sergio Rosini, Livorno (IT); Silvia Trasciatti, Pisa (IT); Mario Petrini, Pisa (IT)

Correspondence Address: VENABLE, BAETJER, HOWARD AND CIVILETTI, LLP, P.O. BOX 34385, WASHINGTON, DC, 20043-9998, US

(73) Assignee: Yissum Research Development Company of the Hebrew University of Jerusalem, Jerusalem (IL)

(21) Appl. No.: 10/766,527

(22) Filed: Jan. 29, 2004

Related U.S. Application Data
(63) Continuation of application No. PCT/IL01/00700, filed on Jul. 29, 2001.

(51) Int. Cl. A61K 38/08; A61K 38/06

(52) U.S. Cl. 514/17; 514/18

(57) ABSTRACT

The present invention relates to a pharmaceutical composition comprising as an effective ingredient an oligopeptide identical or analogous to the C-terminal portion of OGP, having stimulatory activity on the production of hematopoietic cells. Preferred oligopeptides that are used are Tyr-Gly-Phe-Gly-Gly, Tyr-Gly-Phe-His-Gly, Gly-Phe-Gly-Gly or Met-Tyr-Gly-Phe-Gly-Gly. More specifically, these oligopeptides enhance the engraftment of bone marrow transplants, hemopoietic reconstruction, bone marrow re-population and peripheral stem cell mobilization, preferably after chemotherapy or irradiation. The invention further provides methods of treatment and for using these oligopeptides in the preparation of pharmaceutical compositions.

### CROSS-REFERENCE TO RELATED APPLICATION

This is a continuation of International Patent Application No. PCT/IL01/00700 filed Jul. 29, 2001, designating the United States, the disclosure of which is being incorporated herein by reference.

### BACKGROUND OF THE INVENTION

The present invention relates to the use of oligopeptides corresponding to the C-terminal portion of OGP, as stimulators of hematopoiesis. More specifically, these oligopeptides enhance engraftment of bone marrow transplants, hematopoietic reconstruction, bone marrow re-population and number of circulating stem cells, particularly after chemotherapy or irradiation. The invention further provides methods for using these oligopeptides and pharmaceutical compositions comprising them.

Biological and biochemical interactions between bone and bone marrow are far from being fully understood. However recent studies confirm the role of bone marrow derived osteogenic cells in supporting hematopoietic cell development [Teichman, R. S., et al., Hematol. 4:421-426 (2000)].

Bone marrow transplantation studies confirm the bi-directional interactions between the two systems. Bone marrow ablation or iradiation damage triggers an initial local, transient osteogenic reaction [Amsel, S., et al., Anat. Rec. 164:101-111 (1969); Patt, H. M., and Maloney, M. A., Exp. Hematol. 3:135-148 (1975)]. In this osteogenic phase, trabeculae are formed in the marrow cavity. The trabeculae are transient and are resorbed during the reconstitution of haematopoietic marrow. Moreover, in human bone marrow donors, an increase in serum bone formation markers osteocalcin and alkaline phosphatase was recorded after the removal of a substantial portion of iliac bone marrow [Foldes, J., et al., J. Bone Miner. Res. 4:643-646 (1989)]. The hypothesis that human osteoblasts support human hematopoietic progenitor cells is very intriguing: these cells produce factors that directly stimulate the formation of hematopoietic colonies without the addition of exogenously supplied growth factors. In fact, osteoblasts secrete several cytokines including granulocyte colony-stimulating factor (G-CSF), granulocyte-macrophage colony-stimulating factor (GM-CSF), tumor necrosis factor (TNF), and interleukin 6 (IL-6). Besides, cultured osteoblasts support the maintenance of immature phenotype in hematopoietic stem cells [Taichman, et al., Blood 87:518-524 (1996)].

Several of these growth factors improve in vivo bone marrow re-population and peripheral stem cell mobilization after high dose chemotherapy. Among them, G-CSF, GM-CSF, IL-3 (Interleukine-3) and SCF (Stem Cell Factor) have been extensively evaluated [Bungart, B., et al., Br. J. Haematol. 76:174 (1990); Lant, T., et al., Blood 85:275 (1995); Brugger, W., et al., Blood 79:1193 (1992); Molinex, G., et al., Blood 78:961 (1991)] and many others, such as FLT-3, are under study for clinical use [Ashihara, E., et al., Europ. J. Haematol. 60:86 (1998)]. Advances in this field in recent years, have allowed an understanding of several physiological aspects of bone marrow function. Moreover, the ability to modulate differentiation and proliferation of haematological precursors is at the basis of the more innovative therapies such as peripheral blood stem cell transplant, gene transfection and ex vivo expansion of stem cells. In spite of this impressive progress, several aspects of stem cell physiology have not been fully clarified, and several factors, both soluble or cell membrane related, are suspected of being involved in the physiological or pathological proliferation/differentiation of bone marrow cells. The increasing number of agents shown to be able to regulate hematopoiesis supports the critical question regarding the redundancy or subtlety of hematopoietic regulators [Metcaff, D., et al., Blood 82:3515 (1993)].

In addition to the role of classically defined growth factors, several biological agents and cell types could improve or modify both in vivo and ex vivo therapeutic strategies. Human bone marrow-derived endothelial cells support long term proliferation and differentiation of myeloid and megakaryocytic progenitors [Rafii, S., et al., Blood 86:3353 (1995)]; accessory cells may support hematological recovery after bone marrow transplant [Bonnet, D., et al., Bone Marrow Transpl. 23:203 (1991)]; and, even more interesting for present purposes, osteoblasts may enhance the engraftment after HLA-unrelated bone marrow transplant in mice [El-Badri, N. S., et al., Exp. Hematol. 26:110 (1998)].

Many chemical structures have been investigated in order to assess a possible role in bone marrow physiology. For example, effects of glycosaminoglycans have been evaluated both on leukemia-derived cells lines [Volpi, N., et al., Exp. Cell Res. 215:119 (1994)] and in clonogenic tests on human cord blood-derived stem cells [Da Prato, I., et al., Leuk. Res. 23:1015 (1999)]. Even short peptides have been synthesized to reach hemoregulatory and multilineage effects, possibly by enhancement of cytokine production by stromal cells [King, A. G., et al., Exp. Hematol. 20(4):531 (1992); Pelus, L. M., et al., Exp. Hematol. 22:239 (1994)].

Idiopathic myelofibrosis (IMF) is the least common and carries the worst prognosis of the chronic myeloproliferative disorders. The primary pathogenic process is a clonal hematopoietic stem cell disorder which results in anemia, atypical megakaryocyte hyperplasia, splenomegaly and varying degrees of extramedullary hematopoiesis. In contrast, the characteristic stromal proliferation is a reactive phenomenon, resulting from the inappropriate release of megakaryocyte/platelet-derived growth factors, including platelet-derived growth factor (PDGF), transforming growth factor-beta (TGF-beta), basic fibroblast growth factor (bFGF), epidermal growth factor (EGF), and calmodulin [Groopman, J., Ann. Intern. Med. 92:857-858 (1980); Chvapil, M., Life Sci. 16:1345-1361 (1975)]. The median survival of IMF patients is approximately 4 years. Therapeutic strategies in IMF remain predominantly supportive and directed towards the alleviation of symptoms and improvement in quality of life. The most common are blood transfusions, androgens and cytoreductive agents such as hydroxyurea. Bone marrow transplantation is increasingly being taken into consideration, but it still has to be regarded as an experimental approach. Interferon-alpha (IFN-alpha) has shown promising results in early hyperproliferative stages of IMF but has no or only very little effect in more advanced stages of the disease.

It has been previously shown by some of the inventors that osteogenic growth peptide (OGP), a 14-amino acid, highly conserved H4 histone-related peptide, increases blood and bone marrow cellularity and enhances engraftment of bone marrow transplants in mice [Bab, I. A., Clin. Orthop. 313:64 (1995); Gurevitch, O., et al., Blood 88:4719 (1996) and U.S. Pat. No. 5,461,034]. OGP has been isolated from the osteogenic phase of post-ablation bone marrow regeneration [Bab, I., et al., Endocrinology, 128(5);2638 (1991)] and is physiologically present in high abundance in the blood, mainly as a complex with α₂-macroglobulin (α₂-M) [Gavish, H., et al., Biochemistry, 36:14883-14888 (1997)]. Administered in vivo, it enhances bone formation and increases trabecular bone mass; in vitro, it stimulates the proliferation and alkaline phosphatase activity in osteogenic cell lines; in addition, it is mitogenic to fibroblasts [Greenberg, Z., et al., Biochim. Biophys. Acta. 1178:273 (1993)]. In addition to the activity of OGP on bone regeneration, osteoblast activation and fibroblast proliferation, it has been shown to induce, in vivo, a balanced increase in white blood cell (WBC) counts, and overall bone marrow cellularity in mice receiving myeloablative irradiation and syngeneic or semiallogeneic bone marrow transplants [Gurevitch, O., et al., ibid. (1996)].

The C-terminal pentapeptide of OGP, designated OGP(10-14), which seems to be generated by proteolytic cleavage of the full length OGP upon dissociation of the inactive complex with α₂-M, is present in mammalian serum and osteogenic cell cultures at high levels [Bab, I., et al., J. Pept. Res. 54:408 (1999)]. N-terminal modified OGP retains the OGP-like dose-dependent effect on cell proliferation, and it has been suggested that the carboxy-terminal pentapeptide is responsible for the binding to the putative OGP receptor [Greenberg, Z., et al., ibid. (1993)]. Additionally, the inventors have shown previously that in osteogenic MC3T3 E1 cells, mitogenic doses of OGP(10-14), but not OGP, enhance MAP kinase activity in a time- and dose dependent manner. These findings indicate that the OGP(10-14) is responsible for downstream signaling [Gabarin, et al., J. Cell Biol. 81:594-603 (2001)]. It has further been shown that the active form of OGP is its carboxy terminal pentapeptide OGP(10-14). Interestingly, the OGP(10-14) does not form a complex with α₂-M or other OGPBP (OGP binding protein) [Bab, I., J. Peptide Res. 54:408-414 (1999)].

Therefore, the possible hematopoietic activity of synthetic oligopeptides analogous to the C-terminal region of native OGP was evaluated in the present invention. Some such osteogenically active specific peptides are described in U.S. Pat. No. 5,814,610. sOGP(10-14) has been described as having opiate and analgesic activities [Kharchenko et al., Vepr. Med. Khim., 35(2) 106-109, (1989)].

Importantly, the present invention shows that previously known osteogenically active oligopeptides can act as stimulants of the hemopoietic system. For example, the synthetic OGP-derived pentapeptide designated OGP(10-14) has several properties such as increasing blood and bone marrow cellularity in mice, and enhancing engraftment of bone marrow transplants.

This pentapeptide exhibited significant activity on peripheral blood cell recovery after cyclophosphamide (CFA) induced aplasia, and on stem cell mobilization. Furthermore, the ex vivo effect of synthetic OGP(10-14) in bone marrow tissue samples from IMF patients was tested and demonstrated a substantial overall increase in the number of hematopoietic cells. Moreover, the magnitude of the OGP(10-14) effect was directly related to the severity of IMF. These results indicate that OGP(10-14) may stimulate blood cell formation and rescue hematopoiesis.

It is therefore an object of the present invention to use OGP-drived oligopeptides as hematopoietic growth factors. This and other objects of the invention will be elaborated on as the description proceeds.

### SUMMARY OF THE INVENTION

In a first aspect, the invention relates to a pharmaceutical composition comprising as an effective ingredient at least one oligopeptide having stimulatory activity on the production of hematopoietic cells. The oligopeptide used according to the invention has a molecular weight of 200 to 1,000 Da and may be an oligopeptide comprising any of the amino acid sequences Tyr-Gly-Phe-Gly-Gly, Tyr-Gly-Phe-His-Gly, Gly-Phe-Gly-Gly and Met-Tyr-Gly-Phe-Gly-Gly. The pharmaceutical compositions of the invention optionally comprise a pharmaceutically acceptable carrier, diluent or excipient.

In one preferred embodiment of the present aspect, the pharmaceutical composition of the invention comprises an oligopeptide which is a pentapeptide having the formula: Tyr-Gly-Phe-Gly-Gly (designated OGP(10-14)) and a pharmaceutically acceptable carrier.

In another embodiment, the pharmaceutical composition of the invention comprises an oligopeptide which is a pentapeptide having the formula: Tyr-Gly-Phe-His-Gly.

In yet another embodiment, the pharmaceutical composition of the invention comprises an oligopeptide which is a tetrapeptide having the formula: Gly-Phe-Gly-Gly and a pharmaceutically acceptable carrier.

And in a further embodiment, the pharmaceutical composition of the invention comprises an oligopeptide comprising the amino acid sequence Met-Tyr-Gly-Phe-Gly-Gly and a pharmaceutically acceptable carrier, in which the methionine residue is preferably acylated, namely an oligopeptide having the formula: Ac-Met-Tyr-Gly-Phe-Gly-Gly.

The pharmaceutical composition of the invention is intended for enhancement of engraftment of bone marrow transplants, hematopoietic reconstruction, bone marrow re-population and number of circulating stem cells.

In another embodiment the pharmaceutical composition of the invention is intended for enhancement of engraftment of bone marrow transplants, hematopoietic reconstruction, bone marrow re-population and number of circulating stem cells, particularly in patient receiving chemotherapy or irradiation.

The oligopeptide used in the pharmaceutical composition of the invention increases the circulating multilineage progenitor cells percentage. These multilineage progenitor cells are the circulating early precursor CD34 positive cells.

Furthermore, the oligopeptide used as an effective ingredient in the pharmaceutical composition of the invention enhances the immature cell and monocyte recovery and selectively increases any one of the BFU-E and GEMM colony forming units (CFU).

The pharmaceutical composition of the invention is therefore intended for increasing the number of white blood cells (WBC), circulating hematopoietic stem cells as well as overall bone marrow and blood cellularity.

In a specifically preferred embodiment, the composition of the invention is intended for supporting bone marrow transplantation. This effect is due to the activity of the oligopeptides on increasing the number of hematopoietic stem cells, accelerating the hematopoietic reconstruction upon bone marrow transplantation and enhancing the overall cellularity of bone marrow.

According to another specifically preferred embodiment, the pharmaceutical composition of the invention is intended for use in treating bone marrow transplanted subjects suffering from hematological disorders, solid tumors, immunological disorders and/or aplastic anemia. More specifically, the hematological disorders may be lymphomas, leukemias, Hodgkin's diseases and myeloproliferative disorders. Particularly, the myeloproliferative disorder may be idiopathic myelofibrosis (IMF).

In a second aspect, the present invention relates to the use of an oligonucleotide comprising any one of the amino acid sequence Tyr-Gly-Phe-Gly-Gly, Tyr-Gly-Phe-His-Gly, Gly-Phe-Gly-Gly and Met-Tyr-Gly-Phe-Gly-Gly in the preparation of a pharmaceutical composition intended for enhancement of engraftment of a bone marrow transplant, hematopoietic reconstruction, bone marrow re-population and stimulating the number of circulating stem cells.

In a specific embodiment the oligonucleotides of the invention are used in the preparation of a pharmaceutical composition intended for enhancement of engraftment of a bone marrow transplant, hematopoietic reconstruction, bone marrow re-population and number of circulating stem cells, particularly in patient receiving irradiation or chemotherapy.

According to a preferred embodiment, the above specific oligopeptides are used in the preparation of a pharmaceutical composition for increasing the number of circulating multilineage progenitor cells. These multilineage progenitor cells are the circulating early precursor CD34 positive cells.

Furthermore, the oligopeptides used in the preparation of the pharmaceutical composition of the invention enhance the immature cell, monocyte recovery and selectively increase any one of the BFU-E and GEMM colony forming units (CFU).

Accordingly, such oligopeptides may be used in the preparation of pharmaceutical composition intended for increasing the number of white blood cells (WBC), circulating hematopoietic stem cells, and/or overall bone marrow cellularity.

More specifically, the invention provides for the use of these oligopeptides in the preparation of a pharmaceutical composition for supporting bone marrow transplantation. This effect is due to the activity of the oligopeptides on increasing the number of stem cells, accelerating the hematopoietic reconstruction upon bone marrow transplantation and increasing the cellularity of bone marrow.

According to another specifically preferred embodiment, the present invention relates to the use of said oligopeptides in the preparation of a pharmaceutical composition which is intended for treating subjects suffering from hematological disorders, solid tumors, immunological disorders and/or aplastic anemia. More specifically, the hematological disorders may lymphomas, leukemias, Hodgkin's diseases or myeloproliferative disorders, particularly idiopathic myelofibrosis (IMF).

In a third aspect, the present invention provides a method for enhancement of engraftment of a bone marrow transplant, hematopoietic reconstruction, bone marrow re-population and number of circulating stem cells. This method comprises the step of administering to a subject in need thereof, an effective amount of an oligopeptide having stimulatory activity on production of hematopoietic cells as described above, or of the composition of the invention. This method of the invention may be used according to a preferred embodiment for enhancement of engraftment of a bone marrow transplant, hematopoietic reconstruction, bone marrow re-population and number of circulating stem cells in a patient receiving irradiation or chemotherapy.

According to a specific embodiment of this aspect, the invention relates to a method of treating a subject suffering from a hematological disorder, solid tumor, immunological disorder or aplastic anemia. The method of the invention comprises administering to the subject a therapeutically effective amount of an oligopeptide having stimulatory activity on production of hematopoietic cells as described above, or of a composition comprising the same.

In another specific embodiment, this method can be used in support of the treatment of the subject by bone marrow transplantation.

More specifically, the hematological disorders may be lymphomas, leukemias, Hodgkin's disease or myeloproliferative disorders, particularly idiopathic myelofibrosis (IMF).

A preferred embodiment relates to a method for enhancing the number of hematopoietic stem/progenitor cells. According to the invention, this method comprises the steps of exposing these cells to an effective amount of an oligopeptide having stimulatory activity on production of hematopoietic cells as described above, or to a composition comprising the same.

In a specifically preferred embodiment, the method of the invention is intended for enhancing the proliferation of CD34 positive cells.

In one specifically preferred embodiment, the cells are in cell culture and the method may be used ex vivo or in vitro.

Alternatively, the method of the invention may be used as an in vivo method of treatment, preferably of mammals, particularly humans.

The treated subject is one who suffers from, or is susceptible to, decreased blood cell levels, which may be caused by chemotherapy, irradiation therapy, or bone marrow transplantation therapy.

In yet another preferred embodiment, the invention relates to a method for in vitro or ex vivo maintaining and/or expanding hematopoietic stem cells present in a blood sample. This method comprises isolating peripheral blood cells from the blood sample, enriching blood progenitor cells expressing the CD34 antigen, cluttering the enriched blood progenitor cells under suitable conditions, and treating said cells with an oligopeptide having stimulatory activity on production of hematopoietic cells as described above or with a composition comprising the same.

In vivo treatment according to the invention relates to a method for re-populating blood cells in a mammal. This method comprises the steps of administering to said mammal a therapeutically effective amount of an oligopeptide having stimulatory activity on hematopoietic cells as described above, or of a composition comprising the same. These hematopoietic cells may be erythroid, myeloid or lymphoid cells.

### BRIEF DESCRIPTION OF THE DRAWINGS

FIG. 1—A dose dependent effect of pretreatment with sOGP(10-14) on the total number of femoral marrow cells in mice after combined ablative radiotherapy/BMT

OGP(10-14) at the indicated dose was daily injected subcutaneously for 12 days to female C57 BL mice. On day 8 after the onset of OGP(10-14) treatment the mice were subjected to 900 Rad X-ray irradiation, followed by intravenous administration of 10⁵ syngeneic unselected bone marrow cells. On day 14 after the onset of treatment the mice were sacrificed and the femoral bone marrow washed out into phosphate buffered saline. A single cell suspension was prepared by drawing the preparation several times through graded syringe needles. Cell counts were carried out in a hemocytometer. C-control mice given phosphate buffered saline only. Data are mean±SE obtained in at least seven mice per condition. Abbreviations: Fem (femoral), Marr C (marrow cells), D (day), mou (mouse), premed (premedication) stimu (stimulation) and cellu (cellularity).

FIGS. 2A-C—OGP(10-14) stimulates blood cell counts in dose and time dependent manner in mice undergoing chemoablation of hematopoietic tissues Male ICR mice weighing 25 gm each were subjected to chemoablation using cyclophosphamide (CFA), 5 mg/mouse, injected intraperitoneally on days 0 and 1, one injection each day. OGP(10-14) was dissolved in “sterile water for injection” and 0.1 ml of the indicated doses or water only (vehicle) was administered subcutaneously in the nape daily from day −7 to day −1 and from day +2 to day +8. Data are mean±SD obtained in 20 animals per condition. *: significant over CFA+vehicle, p<0.05; **: significant over 1 nmol OGP(10-14) group, p<0.05.

FIG. 2A shows total white blood cell counts.

FIG. 2B shows total monocytes counts.

FIG. 2C shows total immature cells counts.

Abbreviations: cont (control, untreated), veh (vehicle), ce (cell), T (time-days)

FIG. 3—OGP(10-14) stimulates the number of circulating double positive CD34⁺/Sca-1+ in mice undergoing chemoablation of hematopoietic tissues Male ICR mice weighing 25 gm each were subjected to chemoablation using cyclophosphamide (CFA), 5 mg/mouse, injected intraperitoneally on days 0 and 1, one injection each day. OGP(10-14) was dissolved in “sterile water for injection” at 100 nmol/ml concentration and 0.1 ml of this solution or water only (vehicle) was administered subcutaneously in the nape daily from day −7 to day −1 and from day +2 to day +8. CFA ablated mice treated with 10⁶ UI/0.1 ml G-CSF from day +2 to +8 served as positive reference. Data are mean±SD (error bars were too small to be displayed) obtained in 33 animals per condition.

Abbreviations: veh (vehicle), T (time-days), *: significant over CFA+vehicle, p<0.01.

FIGS. 4A-C—Effect of OGP(10-14) treatment regimen on ex vivo colony forming units derived from bone marrow of mice subjected to chemoablation of hematopoietic tissues

Male ICR mice weighing 25 gm each were subjected to chemoablation using cyclophosphamide (CFA), 5 mg/mouse, injected intraperitoneally on days 0 and 1, one injection each day. OGP(10-14) was dissolved in “sterile water for injection” at 100 nmol/ml concentration and 0.1 ml of this solution or water only (vehicle) was administered subcutaneously in the nape daily for the indicated period(s). Bone marrow was harvested on day 9 and analyzed for colony forming units. Data are meam±SD obtained in 10 animals per condition.

FIG. 4A shows CFU-GM.

FIG. 4B shows CFU-GEMM.

FIG. 4C shows BFU-E.

Abbreviations: Colo/di (colonies/dish), Veh (vehicle).

FIGS. 5A-B—Microphotography of bone marrow biopsy

FIG. 5A presents photomicrographs of two parts of bone marrow specimen from idiopatic myelofibrosis (IMF) patient cultured ex vivo for 14 days in the absence of OGP(10-14).

FIG. 5B presents photomicrographs of two parts of bone marrow specimen from idiopatic myelofibrosis (IMF) patient cultured ex vivo for 14 days in the presence of 10⁻⁸ M OGP(10-14). Note increased cell density in specimen cultured with OGP(10-14).

FIGS. 6A-B—Microphotography of bone marrow biopsy

FIG. 6A presents photomicrographs of reticulum stained sections from two parts of bone marrow specimen from idiopatic myelofibrosis (IMF) patient cultured ex vivo for 14 days in the absence of OGP(10-14).

FIG. 6B presents photomicrographs of reticulum stained sections from two parts of bone marrow specimen from idiopatic myelofibrosis (IMF) patient cultured ex vivo for 14 days in the presence of 10⁻⁸ M OGP(10-14). Note normal appearance of OGP(10-14) treated tissue.

FIG. 7—Regression analysis in IMF

Regression analysis in idiopatic myelofibrosis (IMF) patients between hemoglobin level and ex vivo ratio of hematopoietic cells number in OGP(10-14) treated over untreated specimens (T/C ratio), suggesting a direct relationship between the severity of IMF and effect of OGP(10-14). Abbreviations: Hem(hemoglobin), Hemato(hematopoietic), rat(ratio), cellu (cellularity).

### DETAILED DESCRIPTION OF THE INVENTION

A number of methods of the arts of cell biology and peptide chemistry are not detailed herein, being well known to the person of skill in the art. Such methods include peptide synthesis and structural analysis, differential cell counts, cell sorting analyses, colony forming assays, and the like. Textbooks describing such methods are e.g., Current Protocols in Immunology, Coligan et al. (eds), John Wiley & Sons. Inc., New York, N.Y. and Stewart, J. M. and Young J. D., In: Solid Phase Peptide Synthesis, Pierce Chemical Co., Rockford, Ill., pp. 1-175 (1984). These publications are incorporated herein in their entirety by reference. Furthermore, a number of immunological techniques are not in each instance described herein in detail, as they are well known to the person of skill in the art.

The following abbreviations are used herein:
OGP(s)—osteogenic growth polypeptide(s).
OGPBP(s)—osteogenic growth polypeptide binding protein(s).
sOGP—Synthetic OGP.
WBC—(white blood cells).
PBL—(peripheral blood).
CFA—(cyclophosphamide).
BMT—(bone marrow transplantation).
IMF—(idiopatic myelofibrosis).

Several cellular or soluble agents could be responsible for the interaction between bone and bone marrow cells. This interaction seems to be essential for the regulation of commitment, proliferation, and differentiation of hematopoietic stem and progenitor cells.

OGP increases osteogenesis and bone marrow cellularity [Greenberg, Z., et al., ibid. (1993); Gurevitch, O., et al., ibid. (1996)]. Moreover, OGP is a potent mitogen for osteoblastic and fibroblastic cells and bone marrow stromal cells [Greenberg, Z., et al., J. Cellular Biochem, 65:359-367 (1997); Robinson, D., et al, J. Bone Min. Res., 10:690-696 (1995)].

In an osteoblastic cell line, it has recently been reported that OGP activates mitogen-activated protein kinase via a pertussis toxin-sensitive G-protein. These activities appear to be restricted to C-terminal pentapeptide OGP(10-14) and, therefore, it has been suggested that OGP(10-14) is the bioactive form of OGP [Bab, I., et al., ibid. (1999)]. OGP(10-14) could be extremely interesting in view of a possible in vivo utilization, considering the absence of immunogenicity and toxicity and the relative simplicity of the production and handling of the peptide.

Previous studies have demonstrated that after daily s.c. injections of 0.1 to 10 nmol OGP for 2 weeks in normal mice, the peptide induced an increase greater than 50% in the WBC counts and approximately 40% enhancement of overall bone marrow cellularity [Gurevitch, O., et al., ibid., (1996)]. The proportion of different cell types was not altered by the treatment, which suggests a multilineage activity on hematopoiesis. Interestingly, in the experiment described herein, after reversible aplasia induced by the administration of CFA (cyclophosphamide), mice treated by OGP(10-14) recovered faster than those injected with placebo and without any valuable toxicity at the employed doses.

Thus, in a first aspect the present invention relates to a pharmaceutical composition comprising as an effective ingredient at least one oligopeptide having stimulatory activity on the production of hematopoietic cells, preferably having the amino acid sequences Tyr-Gly-Phe-Gly-Gly, Tyr-Gly-Phe-His-Gly, Gly-Phe-Gly-Gly or Met-Tyr-Gly-Phe-Gly-Gly, also denoted by SEQ ID NOs:1, 2, 3, and 4, respectively, and a pharmaceutically acceptable carrier.

The process of blood cell formation whereby red and white blood cells are replaced through the division of cells located in the bone marrow is called hematopoiesis. For review of hematopoiesis see Dexter and Spooncer [Ann. Rev. Cell Biol., 3:423-441 (1987)].

There are many different types of blood cells, which belong to distinct cell lineages. Along each lineage, there are cells at different stages of maturation. Mature blood cells are specialized for different functions. For example, erythrocytes are involved in O₂ and CO₂ transport; T and B lymphocytes are involved in cell and antibody mediated immune responses, respectively; platelets are required for blood clotting; and the granuloctyes and macrophages act as general scavengers and accessory cells. Granulocytes can be further divided into basophils, eosinophils, neutrophils and mast cells.

In a specifically preferred embodiment of the present aspect, the pharmaceutical composition of the invention comprises an oligopeptide which is a pentapeptide having the formula: Tyr-Gly-Phe-Gly-Gly as denoted by SEQ ID NO:1. This pentapeptide is designated OGP(10-14) throughout the present application.

In another embodiment, the pharmaceutical composition of the invention comprises an oligopeptide which is a pentapeptide having the formula: Tyr-Gly-Phe-His-Gly, as denoted by SEQ ID NO:2.

In yet another embodiment, the pharmaceutical composition of the invention comprises an oligopeptide which is a tetrapeptide having the formula: Gly-Phe-Gly-Gly, as denoted by SEQ ID NO:3.

In another embodiment, the pharmaceutical composition of the invention comprises an oligopeptide which is a hexapeptide having the formula Met-Tyr-Gly-Phe-Gly-Gly, as denoted by SEQ ID NO:4, in which the methionine residue may be acylated.

The peptides used as the effective ingredient in the pharmaceutical compositions of the invention are synthetically produced by known organic chemistry methods. Such synthesis is described, for example, in said U.S. Pat. No. 5,814,610.

According to a preferred embodiment of the present aspect, the pharmaceutical composition of the invention is intended for enhancement of engraftment of bone marrow transplants, hematopoietic reconstruction, bone marrow re-population and the number of circulating hematopoietic stem cells.

According to another embodiment, the pharmaceutical composition of the invention is intended for enhancement of engraftment of bone marrow transplants, hematopoietic reconstruction, bone marrow re-population and the number of circulating hematopoietic stem cells of a patient receiving chemotherapy or irradiation.

The capacity of the hematopoietic stem cells to provide for the lifelong production of all blood lineages is accomplished by a balance between the stem cell plasticity, that is the production of committed progenitors cells which generate specific blood lineages, and the stem cell replication in the undifferentiated state (self-renewal). The mechanism regulating hematopoietic stem cell plasticity and self-renewal in vivo have been difficult to define. However, the major contributory factors represent a combination of cell intrinsic and environmental influences [Morrison, et al., Proc. Natl. Acad. Sci. USA 92:10302-10306 (1995)]. The importance of the hematopoietic microenvironment has been established through the use of long-term bone marrow culture systems where hematopoietic cells cultured on stroma allow for the maintenance of HSCs, albeit at low frequencies [Fraser, et al., Proc. Natl. Acad. Sci. USA 89 (1992); Wineman, et al., Blood 81:365-372 (1993)].

The demonstration of hematopoietic cell maintenance in culture has led to efforts to identify candidate ‘stem cell’ factors. The role of hematopoietic cytokines in stem cell maintenance has been studied by direct addition of purified factors to in vitro cultures of stem cell populations followed by transplantation of the cultured cells [Meunch, et al., Blood 81:3463-3473 (1993); Wineman et al., ibid. (1993); Rebel, et al., Blood 83:128-136 (1994)]. Most of the known ‘early-acting’ cytokines such as IL-3, IL-6 and KL have been shown to stimulate proliferation of more committed progenitor cells while concurrently allowing maintenance but not expansion, of cells capable of long-term multilineage repopulation [reviewed in Williams, Blood 81(12):3169-3172 (1993); Muller-Sieburg and Deryugina, Stem Cells, 13:477-486 (1995)]. While these data indicate that the cells plasticity and repopulating function may be preserved by cytokine treatment, the molecules that promote self-renewal of these pluripotent cells remain unknown.

The polypeptide used in the pharmaceutical composition of the invention has been shown to increase the percentage of circulating multilineage progenitor cells. These multilineage progenitor cells are the circulating early precursor CD34 positive cells.

In the human and mouse, primitive mature hematopoietic progenitor cells can be identified a belonging to a class of cells defined by their expression of a cell surface antigen designated CD34. These cells may be referred to as CD34 positive cells. In the mouse, an early subclass of the CD34 positive hematopiotic cells are the double positive CD34+/Sca^{±} cells. The analogous Sca-1 cell surface antigen in the human is Flk2. Therefore, human CD34/ Flk2 double positive cells are considered equivalent to the mouse double positive CD34/ Sca-1 cells.

Human hematopoietic progenitor cells which express the CD34 antigen and/or the Flk 2 receptor are referred to herein as “primitive progenitor cells.” By contrast, hematopiotic cells which do not express either the CD34 antigen or the flk2 receptor are referred to as “mature progenitor cells.” Therefore, as preferred embodiment the multilineage progenitor cells are the circulating early precursor CD34/ Flk2 double positive cells.

As used herein, “progenitor cell” refers to any somatic cell, which has the capacity to generate fully differentiated, functional progeny by differentiation and proliferation. Progenitor cells include progenitors from any tissue or organ system, including, but not limited to, blood, nerve, muscle, skin, gut, bone, kidney, liver, pancreas, thymus, and the like. Progenitor cells are distinguished from “differentiated cells”, which are defined as those cells which may or may not have the capacity to proliferate, i.e., self-replicate, but which are unable to undergo further differentiation to a different cell type under normal physiological conditions. Moreover, progenitor cells are further distinguished from abnormal cells such as cancer cells, especially leukemia cells, which proliferate (self-replicate) but which generally do not further differentiate, despite appearing to be immature or undifferentiated.

Progenitors are defined by their progeny, e.g., granulocyte/macrophage colony-forming progenitor cells (GM-CFU) differentiate into neutrophils or macrophages; primitive erythroid blast-forming units (BFU-E) differentiate into erythroid colony-forming units (CFU-E) which give rise to mature erythrocytes. Similarly, the Meg-CFU, GEMM-CFU, Eos-CFU and Bas-CFU progenitors are able to differentiate into megakaryocytes, granulocytes, macrophage, eosinophls and basophils, respectively.

Various other hematopoitic progenitors have been characterized. For example, hematopoietic progenitor cells include those cells, which are capable of successive cycles of differentiating and proliferating to yield up to eight different mature hematopoietic cells lineages. At the most primitive or undifferentiated end of the hematopietic spectrum, hematopoietic progenitor cells include the hematopietic “stem cells.” These rare cells, which represent 1 in 10,000 to 1 in 100,000 of cells in the bone marrow, each have the capacity to generate >10¹³ mature blood cells of all lineages and are responsible for sustaining blood cell production over the life of an organism. They reside in the bone marrow primarily in a quiescent state and may form identical daughter cells through a process called self-renewal. Accordingly, such an uncommitted progenitor can be described as being “omnipotent,” i.e., both necessary and sufficient for generating all types of mature blood cells.

Progenitor cells which retain a capacity to generate all blood cell lineages, but which cannot self-renew are termed “pluripotent”. Cells which can produce some but not all blood lineages and cannot self-renew are termed “multipotent.”

The oligopeptides used in the invention are useful in preserving any of these progenitors cells, including unipotent progenitor cells, pluripotent progenitor cells, and/or omnipotent progenitor cells. The oligopeptides, and particularly OGP(10- 14), demonstrate particular efficacy in preserving hematopoietic progenitor cells.

In a further preferred embodiment, the oligopeptide used as an effective ingredient in the pharmaceutical composition of the invention enhances the immature cell monocyte recovery and selectively increases any one of the BFU-E and GEMM colony forming units (CFU).

Example 3 below describes ex vivo assessment of hematopoietic colony formation derived from OGP(10-14) and control treated mice. The results indicate an increase of GEMM-CFU and BFU-E in cultures derived from OGP(10-14)-treated mice compared to the vehicle only control group, whereas a positive G-CSF control induces a significant increase of GM-CFU. The increases in colony formation in cultures derived from OGP(10-14) treated mice where apparent only when the treatment began seven days prior to chemoablation. Both in vivo and ex vivo results reached by OGP(10-14) confirm the previously reported multilineage activity of full-length OGP compared to different cytokines. Differently to other growth and mobilizing factors [Fleming, W., et al., Proc. Natl. Acad. Sci. USA 90:3760 (1993)], sOGP(10-14) increases the number of hematopoeitic stems cells in the peripheral blood without reducing the bone marrow stem cells compartment.

The pharmaceutical composition of the invention may therefore be intended for increasing the number of white blood cells (WBC), circulating hematopoietic stem cells, and overall bone marrow cellularity.

In a specifically preferred embodiment, the composition of the invention is intended for supporting bone marrow transplantation. This effect is due to the activity of the oligopeptides that increases the number of stem cells, accelerates the hematopoietic reconstruction upon bone marrow transplantation and increases the cellularity of bone marrow.

As described in Example 1, the oligopeptides of the invention have been found to enhance the engraftment of bone marrow transplants and to stimulate hematopoietic reconstruction. Bone marrow transplantation (BMT) is progressively and rapidly becoming the treatment of choice in instances of hematological malignancies such as lymphomas, Hodgkin's diseases and acute leukemia as well as solid cancers, in particular melanoma and breast cancer. Recently, BMT is increasingly being taken into consideration in treatment of myeloproliferative disorders such as IMF (idiopathic myelofibrosis). Potentially, with improved methods, BMT can also be used for treating other catastrophic diseases—AIDS, aplastic anemia and autiommune disorders. The aim of all BMT is to replace the host hematopoietic stem cells, omnipotent and pluripotent, injured by chemotherapy, radiation or disease. These stem cells can replicate repeatedly and differentiate to give rise to the whole variety of cells present in blood namely erythrocytes, platelets and WBC which include lymphocytes, monoccytes and neutrophils. Resident macrophages and osteoclasts are also derived from hemopoietic omnipotent stem cells. As the stem cells differentiate, they commit themselves more and more to a particular lineage until they can form only one kind of the above cells.

Therefore, according to another specifically preferred embodiment, the pharmaceutical composition of the invention may be used in treating bone marrow transplanted subjects suffering from a hematological disorder, solid tumor, immunological disorder or aplastic anemia. More specifically, the hematological disorder may be a lymphoma, Hodgkin's disease or acute leukemia and myeloproliferative disorder, particularly idiopathic myelofibrosis (IMF).

In IMF bone erythropoiesis evolves to a progressive failure, whereas ectopic hemopoiesis develops and increases. Pathological calcification of fibrosis and structural alterations of trabecular bone may be responsible for an absolute or relative deficit of osteoblasts secreted factors and thus, at least partially responsible for the impaired bone marrow function.

The results described in Example 4, strongly suggest that the OGP(10-14) can increase the hematopoietic cell density of bone marrow in cultured bone fragments from IMF patients without modifying, in such a short time, the fibrosis. The cell increment appears to be balanced and it does not account for the expansion of atypical cells. One may not exclude, of course, that OGP(10-14) simply preserves in culture the bone marrow structure and cellularity of IMF samples compared to those found in samples cultured without the pentapeptide. However, the preserved or even increased cellularity in some OGP(10-14) cultured samples compared to that found in the native ones, suggests a proliferative activity of the peptide. It is not clear, at present, if OGP acts on blood precursors directly or via stromal cells or different cell populations but, at least at the morphological level, its activity appears independent of a significant remodelling of microenvironment.

One implication of this observation is that OGP(10-14) is, in fact, able to enhance, in vitro, three lineage expansion of human hematopoietic cells.

The pharmaceutical compositions of the invention comprise as active ingredient an oligopeptide as described above, or a mixture of such oligopeptides, in a pharmaceutically acceptable carrier, excipient or stabilizer, and optionally other therapeutic constituents. Acceptable carriers, excipients or stabilizers are non-toxic to recipients at the dosages and concentrations employed, and include buffers, such as phosphate buffered saline and like physiologically acceptable buffers, and more generally all suitable carriers, excipients and stabilizers known in the art, e.g., for the purposes of adding flavors, colors, lubrication, or the like to the pharmaceutical composition.

Carriers may include starch and derivatives thereof, cellulose and derivatives thereof, e.g., microcrystalline cellulose, Xantham gum, and the like. Lubricants may include hydrogenated castor oil and the like.

A preferred buffering agent is phosphate-buffered saline solution (PBS), which solution is also adjusted for osmolarity.

A preferred pharmaceutical formulation is one lacking a carrier. Such formulations are preferably used for administration by injection, including intravenous injection.

The preparation of pharmaceutical compositions is well known in the art and has been described in many articles and textbooks, see e.g., Remington's Pharmaceutical Sciences, Gennaro A. R. ed., Mack Publishing Company, Easton, Pa., 1990, and especially pages 1521-1712 therein.

The pharmaceutical compositions of the invention can be prepared in dosage units forms. The dosage forms may also include sustained release devices. The compositions may be prepared by any of the methods well known in the art of pharmacy. Such dosage forms encompass physiologically acceptable carriers that are inherently non-toxic and non-therapeutic. Examples of such carriers include ion exchangers, alumina, aluminum stearate, lectithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose- based substances, and PEG. Carriers for topical or gel-based forms of these polypeptides include polysaccharides such as sodium carboxymethylcellulose or methylcelluslose, polyvinylpyrrolidone, polyacrylasts, polyoxyethylene-block polymers, PEG, and wood was alcohols. For all adminstratioins, conventional depot forms are suitably used. Such forms include for example, microcapsules, nano-capsules, liposomes, plasters, inhalation forms, nose sprays, sublingual tablets, and sustained-release preparations.

Suitable examples of sustained-release preparations include semi-permeable matrices of solid hydrophobic polymers containing the oligopeptides according to the invention, which matrices are in the form of shaped articles, e.g. films, or micro-capsules. Examples of sustained-release matrices include polyesters, hydrogels, polylactides as described by, (U.S. Pat. No. 3,377,919), copolymers of L-glumatic acid and y-ethyl-L-glutamate, non-degradable ethylene-vinyl acetate, degradable lactic acid-glycolic acid copolymers such as the Lupron Depots™ (injectable microspheres composed of lactic acid-glycolic acid copolymer and leuprolide acitate), and poly-D-(-)3-hydroxybutyic acid. While polymers such as ethylenevinyl acetate and lactic acid-glycolic acid enable release of molecules for over 100 days, certain hydrogels release proteins for shorter time periods. When encapsulated, the peptides remain in the body for a long time, they may denature or aggregate as a result of exposure to moisture of 37° C., resulting in a loss of biological activity and possible changes in immunogenicity. Rational strategies can be devised for stabilization depending on the mechanism involved. For example, if the aggrergation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release oligopeptides and particularly the sOGP1-14 compositions also include lipsomally entrapped polypeptides. Lipsomes containing these polypeptides are prepared by methods known in the art, such as described in Eppstein, et al., Proc. Natl. Acad. Sci. USA 82:3688-3692 (1985); Hwang, et al., Proc. Natl. Acad. Sci. USA 77:4030 (1980); U.S. Pat. Nos. 4,485,045 and 4,544,545. Ordinarily, the lipsomes are the small (about 200-800 Angstroms) unilamelar type in which the lipid content is greater than about 30 mol. % □□ cholesterol, the selected proportion being adjusted for the optimal polypeptides therapy. Lipsomes with enhanced circulation time are disclosed in U.S. Pat. No. 5,013,556.

Therapeutic formulations of the oligopeptides are prepared for storage by mixing these polypeptide having the desired degree of purity with optional physiologically acceptable carriers, excipients, or stabilizers [Remington's Pharmaceutical Sciences, 16h edition, Osol, A., Ed., (1980)], in the form of lyophilized cake or aqueous solutions. Acceptable carriers, excipients, or stabilizers are non-toxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbhydrates including glucose, mannose, or dextrins; chelating agents such as EDTA; sugar alcohols such as mannitol and sorbitol; slat-forming counter-ions such a sodium; and/or non-ionic surfactants such as Tween, Pluronics™ or polyethlene glycol (PEG).

The oligopeptides may also be entrapped in micro-capsules prepared, for example, by coacervation techinques or by interfacial polymerization (for example, hydroxymethylcellulose or gelatine-microcapsules and poly-(methylmethacylate) microcapsules, respectively), in colloidal drug delivery system (for example, liposomes, albumin microshperes, microemulsions, nanoparticles, and nanocapsules), or in macroemulsions. Such techniques are disclosed in Remington's Pharmaceutical Sciences, ibid.

The pharmaceutical composition is preferably for a once daily use by a subject in need, and preferably comprises a dosage of active ingredient of about 0.001 to about 50 nmol, more preferably about 0.05 to 25 nmol, most preferably about 0.1 to about 10 nmol.

It is to be appreciated that in addition to the described oligopeptides, the transplantation-supporting composition of the present invention may further optionally comprise other therapeutic constituents. Such constituents may be one or more known cytokines, for example, IL-3, IL-4, IL-5, G-CSF, GM-CSF (granulocytemacrophage colony stimulating factor) and M-CSF (macrophage colony stimulating factor). When such further component is incorporated in the composition, the effect of the composition in supporting bone-marrow transplantation can increase synergistically.

As a second aspect, the present invention relates to the use of any of the above described oligopeptides, particularly Tyr-Gly-Phe-Gly-Gly, Tyr-Gly-Phe-His-Gly, Gly-Phe-Gly-Gly and Met-Tyr-Gly-Phe-Gly-Gly, as denoted by SEQ ID NOs:1, 2, 3, and 4, respectively, in the preparation of a pharmaceutical composition for enhancement of engraftment of bone marrow transplant, hematopoietic reconstruction, bone marrow re-population and number of circulating stem cells.

In addition, the oligopeptides described herein may be used in the preparation of pharmaceutical compositions for accelerating the engraftment of bone marrow transplants, enhancing proliferation of transplanted stem cells and thus increasing the availability of all types of hematopoietic cells including erythrocytes and thus obviating the need for supporting the host with these cells for at least several weeks; enhancing stromal hematopoietic microenvironment by increasing the stromal cells number and/or expression of stromal cell derived factors that support hemopoiesis; enhancing the hematopoietic stem cell expression of receptors to factors that support hemopoiesis; enhancing the “homing” of intravenously administered bone marrow transplants to the host bone marrow; enhancing the restoration of blood cellularity after BMT; enabling successful transplantation using reduced cell number, thus decreasing the number of (multiple) marrow extractions from donors and enabling the use of transplants as small as 10-15 ml (instead of 1000 ml); increasing the number of hematopoietic omnipotent and/or pluripotent stem cells in the donor peripheral blood, thus improving the feasibility of transplanting stem cells from peripheral blood; increasing the number of hematopoietic stem cells in vitro in long-term bone marrow cultures for use as transplants and also providing for a method of inhibiting growth of tumor cells in allografts from leukemia patients; enhancing the endogenous restoration of marrow and blood cellularity after chemo- and/or radiotherapy; and enhancing the restoration of population of resident macrophages after BMT or after chemo- and/or radiotherapy.

The magnitude of a therapeutic dose of an oligopeptides or the composition of the invention will of course vary with the group of patients (age, sex, etc.), the nature of the condition to be treated and with the particular oligopeptide employed and its route of administration. In any case the therapeutic dose will be determined by the attending physician.

Any suitable route of administration may be employed for providing a mammal, especially a human, with an effective dosage of a polypeptide of this invention. Intravenous, subcutaneous and oral administration may be preferred.

As a preferred embodiment, these oligopeptides are used for the preparation of a pharmaceutical composition for increasing the circulating multilineage progenitor cells percentage. These multilineage progenitor cells are the circulating early precursor CD34 positive cells, and preferably, CD34/Flk2 double positive cells.

A “hematopoietic stem/progenitor cell” or “primitive hematopoietic cell” as described above, is a cell which is able to differentiate to form a more committed or mature blood cell type. A “hematopoietic stem cell” or “stem cell” is one that is specifically capable of long-term engraftment of a lethally irradiated host.

A “CD34⁺ cell population” is enriched for hemotopoietic stem cells. A CD34⁺ cell population can be obtained from umbilical blood or bone marrow, for example. Human umbilical cord blood CD34⁺ cells can be selected for using immunomagnetic beads sold by Miltenyi (Calfornia), following the Manufacturer's directions.

Furthermore, the oligopeptides used for the preparation of the pharmaceutical composition of the invention enhance the immature cell and monocyte recovery and selectively increases any one of the BFU-E and GEMM colony forming units (CFU).

Accordingly, such oligopeptides are used in the preparation of the pharmaceutical composition for increasing the number of white blood cells (WBC), circulating hematopoietic stem, and overall bone marrow cellularity.

More specifically, the invention provides the use of these polypeptides in the preparation of a pharmaceutical composition for supporting bone marrow transplantation. This effect is due to the activity of the oligopeptides in increasing the number of stem cells, accelerating the hematological reconstruction upon bone marrow transplantation and increasing the cellularity of bone marrow.

According to another specifically preferred embodiment, the present invention relates to the use of said oligopeptides in the preparation of a pharmaceutical composition for treating a subject suffering from hematological disorders, solid tumors, immunological disorders and aplastic anemia. More specifically, the hematological disorder may be a lymphoma, leukemias, Hodgkin's disease and myeloproliferative disorders, particularly idiopathic myelofibrosis (IMF).

In a third aspect, the present invention provides a method for enhancement of engraftment of bone marrow transplant, hematopoietic reconstruction, bone marrow re-population and number of circulating stem cells. This method comprises administering to a subject in need thereof, an effective amount of an oligopeptide having stimulatory activity on hematopoietic cells as described above, or of a composition of the invention.

According to another embodiment, the invention provids a method for enhancement of engraftment of bone marrow transplant, hematopoietic reconstruction, bone marrow re-population and number of circulating stem cells in patients receiving chemotherapy or irradiation.

In yet another embodiment, an effective amount of the oligopeptides or the composition of the invention may be used to improve engraftment in bone marrow transplantation or to stimulate mobilization and/or expansion of hematopoietic stem cells in a mammal prior to harvesting hematopoietic progenitors from the peripheral blood thereof.

According to a specific embodiment of this aspect, the invention relates to a method of treating a subject suffering from a hematological disorder, solid tumor, immunological disorder or aplastic anemia, by administering to the subject a therapeutically effective amount of an oligopeptide having stimulatory activity on production of hematopoietic cells, or of a composition comprising the same according to the invention.

In another specific embodiment, this method can be used in support of the treatment of the subject by bone marrow transplantation.

For therapeutic applications, the oligopeptides or the pharmaceutical composition useful according to the invention are administered to a mammal, preferable a human, in a physiologically acceptable dosage from, including those that may be administered to a human intravenously as a bolus or by continuous infusion over a period of time. Alternative routes of administration include intramuscular, intraperitoneal, intra-cerebrospinal, subcutaneous, intra-articular, intrasynovial, intrathecal, oral or topical routes. The oligopeptides or the compositions of the invention also are suitably administered by intratumoral, peritumoral, intralesional, or perilesional routes or to the lymph, to exert local as well as systemic therapeutic effects.

The oligopeptides or the pharmaceutical compositions to be used for in vivo administration must be sterile, This is readily accomplished by filtration through sterile filtration membranes, prior to or following lypophillization and reconstitution. Oligopeptides may be stored in solution. Therapeutic oligopeptides compositions generally are placed, into a container having a sterile access port, for example, an intravenous solution bag or vial having a stopper pierceable by a hypodermic injection needle.

An “effective amount” of any of the oligopeptides or compositions of the invention to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer the oligopeptide until a dosage is reached that achieves the desired effect. A typical daily dosage for systemic treatment might range from about 0.001 nmol/Kg to up to 50 nmol/Kg or more, depending on the factors mentioned above.

Another specific embodiment relates to the treatment of a subject carrying a transplant, where an ex vivo method may be adopted. In this method, the cells intended for transplantation are exposed to effective amount of the oligopeptides or compiositions of the invention, prior to their transplantation.

The most common way currently available for acquiring a sufficient amount of hematopoietic stem cells for transplantation is to extract 1 liter or more of marrow tissue from multiple site in the donor's bones with needle and syringe, an involved process that usually requires general anaesthesia. The donors of allogeneic BMT are usually siblings whose tissue types are compatible and sometimes unrelated donors who are matched to the recipient by HLA typing. Autologous transplants, that eliminate the need for HLA matching may be used in patients undergoing ablative chemoradiotherapy for the eradication of solid tumors. Autologous stem cells may also be obtained from the umbilical cord blood at birth and stored for future administration.

After transplantation and prior to the establishment of a donor-derived functioning marrow the patients hosting BMT present with a transient marked pancytopenia that exposes them to infections. The incidence of bacterial and fungal infections correlates with both the severity and duration of pancytopenia [Slavin, S. and Nagler, A., Transplantation (1992)]. For a similar reason, the CSF fail to support erythropoiesis and platelet formation. Oligopeptides that support hematopoiesis may prove useful in other ways as well. Some investigators have found that adding stem cells from the peripheral blood to those from the bone marrow significantly increases the rate of engraftment extracting sufficient numbers of stem cells from peripheral blood is a complicated procedure. Administering such oligopeptides to donors to increase the number of stem cells in the blood will improve the feasibility of transplanting stem cells from peripheral blood [Golde, D. W., Sci. Am. 36 December (1991)].

The prerequisite for hematopoiesis and therefore successful MBT is the presence of functional stromal cells and tissue that compromise the hematopoietic microenvironment, determine the homing of the injected stem cells from the circulation to the bone marrow and support hematopoiesis [Watson, J. D. and McKenna, H. J. Int. J. Cell Cloning 10:144 (1992)]. Bone marrow derived stromal tissue also provide the conditions to sustain stem cells in in vitro long-term bone marrow cultures. At present this technology suffices to keep stem cells alive. Adding the appropriate hemopoietic oligopeptides to these cultures may help expand the stem cell population in vitro, this providing increased numbers of these cells for transplantation.

A combined in vitro/in-vivo approach may provide the basis for a forward-looking strategy for (i) obtaining small stem cell preparations from donor blood or marrow and (ii) healthy individuals to have their stem cells stored for a time when the cells might be needed to treat a serious disease, thus bypassing the complexity associated with the use of allogeneic BMT.

It would therefore be of therapeutic importance to use small peptides such as the oligopeptides described in the present application, that stimulate post-BMT hematopoietic reconstruction by enchancing in vivo, ex vivo and/or in vitro the hematopoietic microenvironment of which fibrous tissue, bone and bone cells are important components. Such peptides may also support hematopoiesis in spontaneously occurring or induced myelosuppression condition that do not necessarily involved BMT.

The oligopeptides described in the present application and preferably the pentapeptide OGP(10-14), appear to directly act at the level of the early hematopoietic precursor (i.e., hematopoietic stem/progenitor cells). Such an expanded stem cell population can serve as the source of cells for myelopoiesis, erythropoiesis (e.g., splenic erythropoiesis) and lymphopoiesis. Accordingly, these oligopeptides can be used to stimulate proliferation and/or maintenance of hematopoietic stem/progenitor cells either in vitro or in vivo (e.g., for treating hematopoietic diseases or disorders).

Therefore, a preferred embodiment relates to a method for enhancing the proliferation of hematopoietic stem/progenitor cells. According to the invention, this method comprises the steps of exposing these cells to an effective amount of an oligopeptide having stimulatory activity on hematopoietic cells, or to an effective amount of a composition comprising the same, as described above. According to the invention such exposure is effective in enhancing the proliferation of said cells.

The term “enhancing proliferation of a cell” encompasses the step of increasing the extent of growth an/or reproduction of the cell relative to an untreated cell either in vitro or in vivo. An increase in cell proliferation in cell culture can be detected by counting the number of cells before and after exposure to a molecule of interest. The extent of proliferation can be quantified via microscopic examination of the degree of confluency. Cell proliferation can also be quantified using a thymidine or BrdU incorporation assay.

In a specifically preferred embodiment, the method of the invention is intended for enhancing the proliferation of a CD34 positive cells, preferably Flk2 positive cells.

The oligopeptides or the compositions of the invention are useful in in vivo or ex vivo enhancing the number and/or proliferation and/or differentiation and/or maintenance of hematopietic stem/progenitor cells, expand population of these cells and enhance repopulation of such cells and blood cells of multiple lineages in a mammal.

In one specifically preferred embodiment, these cells are in cell culture and therefore, this would be an ex-vivo/in vitro method.

Alternatively, the method of the invention may be used as an in vivo method of treatment, in case that the treated cells are present in a mammal.

“Treatment” refers to both therapeutic treatment and prophylactic or preventative measures. Those in need of treatment include those already with the disease or disorder as well as those in which the disease or disorder is to be prevented.

“Mammal” for purposes of treatment refers to any animal classified as a mammal including, human, domestic and farm animals, and zoo, sports, or pet animals, such as dogs, horses, cats, cows, etc. Preferably, the mammal is human.

In a specific embodiment, the mammal treated by the method of the invention is suffering from, or is susceptible to, decreased blood cell levels, which may be caused by chemotherapy, irradiation therapy, bone marrow transplantation therapy or any other iatrogenic or natural cause.

Chemo- and radiation therapies cause dramatic reductions in blood cell population in cancer patients. At least 500,000 cancer patients undergo chemotherapy and radiation therapy in the US and Europe each year and another 200,000 in Japan. Bone marrow transplantation therapy of value in aplastic anemia, primary immunodeficiency, acute leukemia and solid tumors (following total body irradiation) is becoming more widely practiced by the medical community. At least 15,000 Americans have bone marrow transplants each year. Other diseases can cause a reduction in entire or selected blood cell lineages. Examples of these conditions include anemia (including macrocytic and aplactic anemia); thrombocytopenia; hypoplasia; immune (autoimmune) thrombocytopenic purpur (ITP); and HIV induced ITP.

Pharmaceutical products are needed which are able to enhance reconstitution of blood cell populations of these patients.

Accordingly, it is an object of the present invention to provide a method for enhancing the proliferation and/or differentiation and/or maintenance of primitive hematopoietic cells. Such a method may be useful for enhancing repopulation of hematopoietic stem cells and thus mature blood cell lineages. This is desirable where a mammal has suffered a decrease in hematopoietic or mature blood cells as a consequence of disease, radiation or chemotherapy. This method is also useful for generating expanded populations of such stem cells and mature blood cell lineages from such hematopoietic cells ex vivo.

In yet another preferred embodiment, the invention relates to a method for in vitro/ex-vivo maintaining and/or expanding stem cells. This method comprising isolating peripheral blood cells from a blood sample, enriching blood progenitor cells expressing the CD34 antigen, cluttering the enriched blood progenitor cells under suitable conditions, and treating said cells with an oligopeptide having stimulatory activity on hematopoietic cells, or with a composition comprising as an effective ingredient an oligopeptide having stimulatory activity on hematopoietic cells, according to the invention.

In a specific embodiment, the method of the invention might include a further step of exposing the treated cells to a cytokine. As a non-limiting example, such cytokine may be selected from the group consisting of TPO (Thrombopoietin), EPO (Erythropoietin), M-CSF (Macrophage-colony stimulating factor), GM-CSF (Granulocyte-macrophage-CSF), G-CSF (Granulocyte CSF), IL-1 (Interleukin-1), IL-2, IL-3, IL-4, IL-5, IL-6, IL-7, IL-8, IL-9, IL-10, IL-12, LIF (Leukemia inhibitory factor) and KL (Kit ligand).

As an embodiment to an in vivo treatment, the invention relates to a method for re-populating blood cells in a mammal. This method comprises the steps of administering to said mammal a therapeutically effective amount of an oligopeptide having stimulatory activity on hematopoietic cells, or of an effective amount of the composition of the invention. These hematopoietic cells may be any one of erythroid, myeloid and lymphoid cells.

“Lymphoid blood cell lineages” are those hematopoietic precursor cells which can differentiate to form lymphocytes (B-cells or T-cells). Likewise, “lymphopoiesis” is the formation of lymphocytes.

“Erythroid blood cell lineages” are those hematopoietic precursor cells which candifferentiate to form erythrocytes (red blood cells) and “erythropoiesis” is the formation of erythrocytes.

The phrase “myeloid blood cell lineages”, for the purposes herein, encompasses all hematopoietic precursor cells, other than lymphoid and erythroid blood cell lineages as defined above, and “myelopoiesis” involves the formation of blood cells (other than lymphocytes and erythrocytes).

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Reagents

1. C-terminal pentapeptide of Osteogenic Growth Peptide(10-14) [sOGP(10-14)]: Tyr-Gly-Phe-Gly-Gly; M. W. 499.7 (SEQ ID NO:1) was supplied by Polypeptides Laboratories Inc. (Torrance, Calif. 90503, USA Batch No.9712-006).
2. CFA—cyclophosphamide (CFA, SIGMA, 5 mg/mouse), was used for induction of marrow ablation.
3. Dexter-like medium: McCoy's Medium (Gibco-Life technologies, USA) with 12.5% fetal bovine serum (FBS, Hyclone, Holland), 12.5% horse serum (HS, Sigma, St Louis, Mo.), 0.8% essential and 0.4% non essential aminoacids (Gibco-Life technologies, USA), 1% glutamine (Sigma, St Louis, Mo.), 0.4% vitamins including choline, folic acid, inositol, nicotinamide, pyridoxal HCl, riboflavin, thiamine HCl, D-Ca pantothenate (Gibco-Life technologies, USA), 1% amphotericine B (Fungizone, Bristol-Myers Squibb), 1% gentamicine, and 10⁻⁶ M hydrocortisone in presence of recombinant human stem cell factor (50 ng/mL rhSCF, Calbiochem, USA), recombinant human granulocyte-monocyte colony stimulating factor (rhGM-CSF 10 ng/mL, Sandoz, Switzerland), recombinant human interleukine-3 (rhIL-3 10 ng/mL, Calbiochem, USA) and recombinant human erythropoietin (rhEpo 2 units/mL, Sigma, St Louis, Mo.) with or without sOGP(10-14) 10⁻⁸M (Abiogen Pharma SpA Research Laboratories).
4. E.D.T.A acid buffer (Mielodec, Bio Optica, Milan, Italy).
   Animals

ICR male mice were purchased from Charles River's (Italy) and maintained under specific pathogen-free conditions.

CV57 Black female mice were from the animal facility of the Hebrew University Medical School (Jerusalem, Israel). The mice of either strain weighed 25 g on their arrival at the inventors' laboratory.

Statistical Analysis Comparisons between groups were made using a Fisher's PLSD, factorial or for repeated measures, analysis of variance (ANOVA). Mann-Whitney test was used, for colony assays.

### Example 1

Effect of OGP(10-14) on engraftment of bone marrow transplant

Materials and Methods

CV57 Black female mice were used to investigate the possible effect of OGP(10-14) on engraftment of bone marrow transplats. OGP(10-14) in phosphate buffered saline was administered by daily subcutaneous, 10 μl injections for 12 days. The daily dose ranged from 0.001 to 10 nmol per mouse. Control mice received phosphate buffered saline only. On day 8 after the onset of OGP(10-14) treatment the mice were subjected to total body X-ray irradiation consisting of a single 900 rad dose using a ⁶⁰Co source (Picker C-9, 102.5 rad/min). This was followed immediately by an intravenous injection of 10⁵ unselected syngeneic bone marow cells. The animals were sacrificed 14 days after the onset of OGP(10-14) treatment, both femurs were dissected out and their ephiphyseal ends removed. The bone marrow was washed out completely into phosphate buffered saline (PBS). A single cell suspension was prepared by drawing the preparation several times through graded syringe needles and the cells were counted in a hemocytometer.

Results

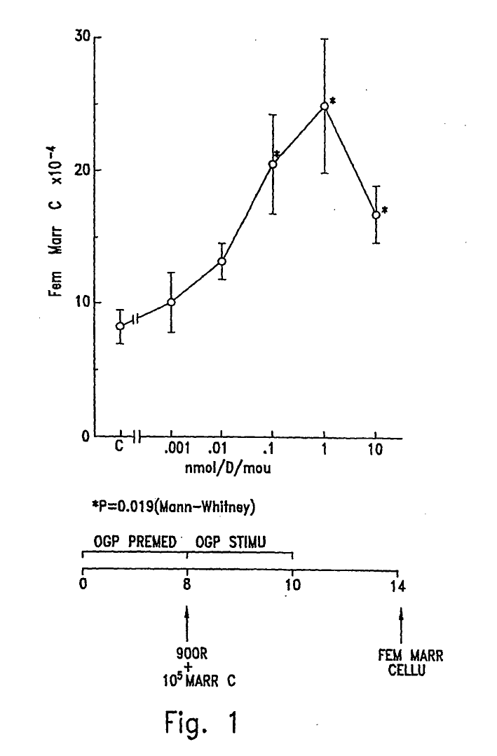

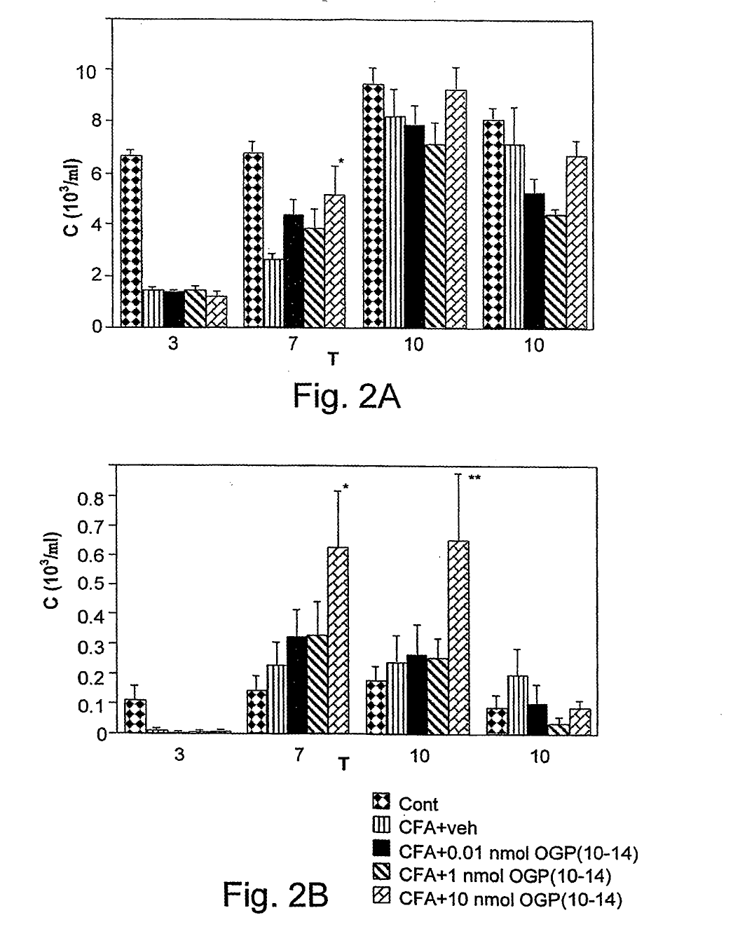

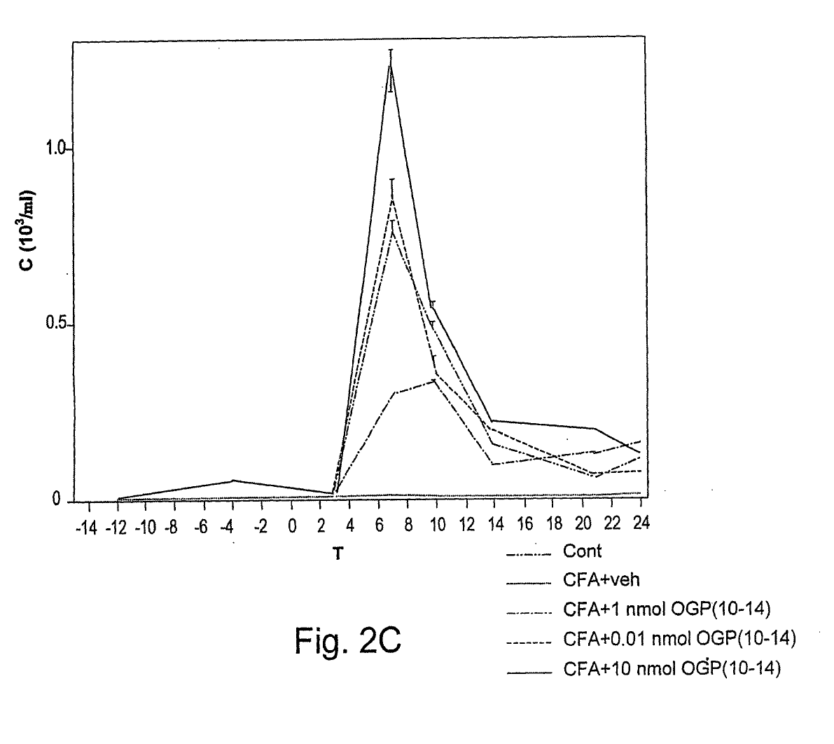

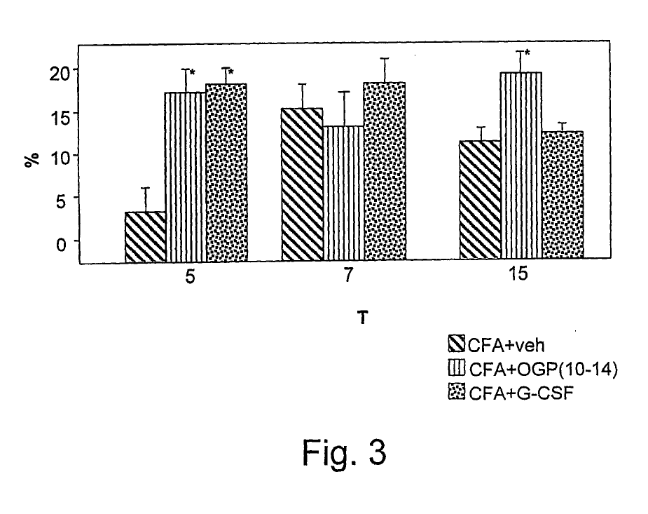

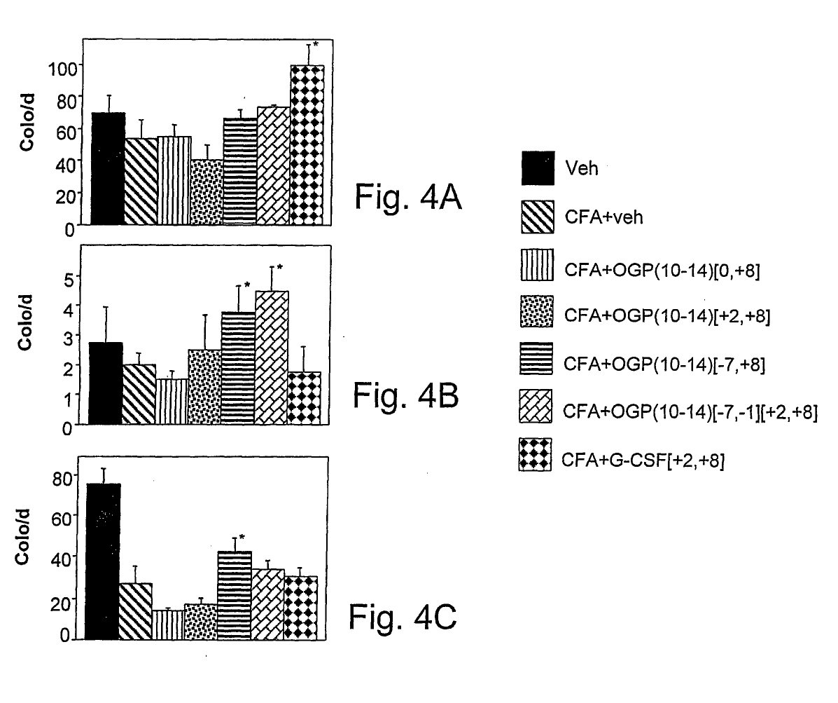

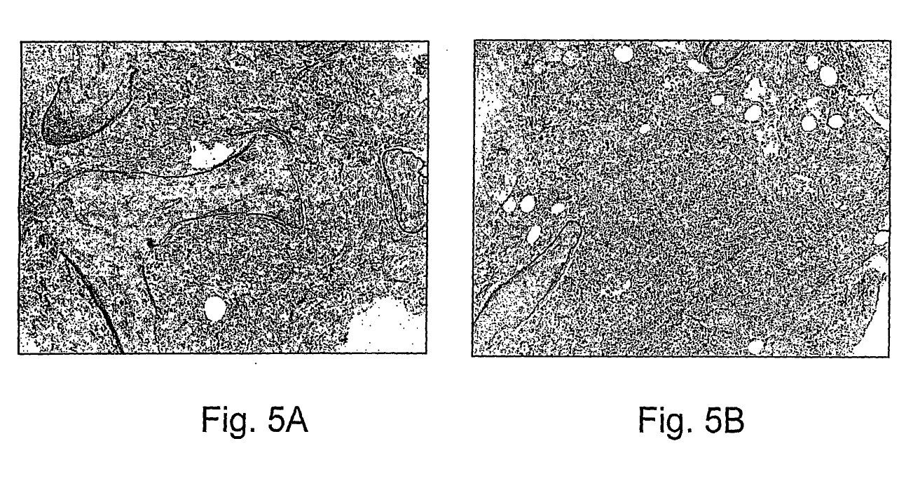

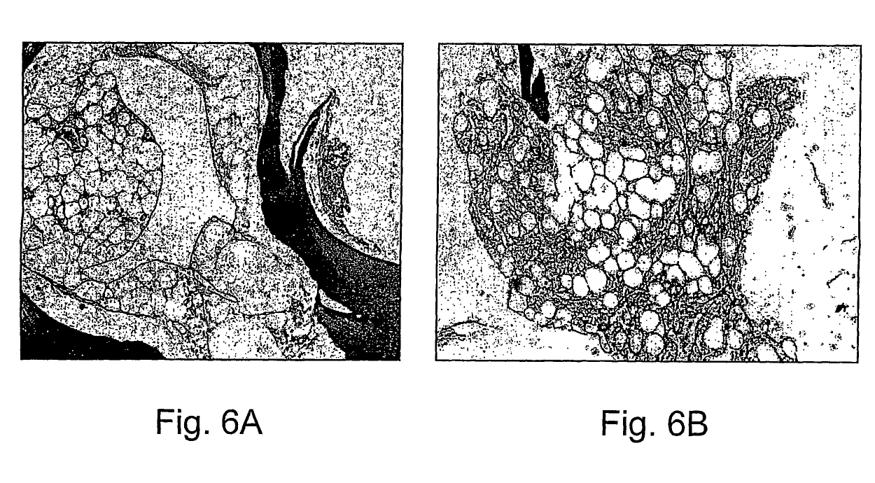

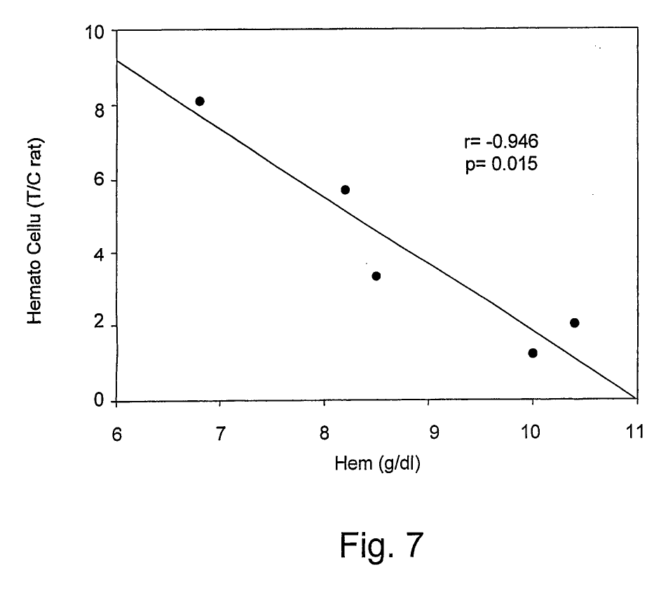

FIG. 1 shows a stimulatory effect of the OGP(10-14) on the number of post-irradiation/post-transplantation total femoral bone marrow cells. This effect was dose dependent showing, at the three highest doses, statistically significant, 2-fold increase in cell counts over the PBS controls.

### Example 2

Evaluation of the OGP(10-14) Toxicity

A shown above, the OGP(10-14) has been found to enhance the engraftment of bone marrow transplants. Threfore, prior to further, detailed analysis of the pharmacological activity of said peptide, the possible toxicity of said peptide was next evaluated.

Fifty-five mice were evaluated for possible OGP(10-14)-related toxicity after 15 days of subcutaneous administration, at the dose of 10 nmol/mouse, and results were compared to those obtained in 30 placebo-treated controls. No differences were found concerning survival, behaviour, body weight gain and gross examination. Concerning the haematological parameters, administration of the reported doses of peptide did not induce any significant modifications in the number of white blood cells (WBC), red blood cells (RBC), platelets (PLT) or haemoglobin (Hb) level.

### Example 3

OGP(10-14) Stimulates Hematopoietic Recovery After Bone Marrow Chemoablation

Materials and Methods

In this set of experiments bone marrow ablation was induced by an intraperitoneal injection of cyclophophamide (CFA, SIGMA, 5 mg/mouse in 150 (1 sterile PBS) for two consecutive days (designated “day 0” and “day 1”. This protocol has been demonstarted to induce severe, reversible leucopoena with L. D. <30 [Spangrude, G. J. et al, Science, 241:58 (1988)]. The lowest bone marrow cell counts were recorded six days after the first injection.

To evaluate the effect of OGP(10-14) on WBC differential cell counts and determine the OGP(10-14) “dose of choice” to be used in further experiments, mice were treated daily by subcutaneous injections of 0.1 ml OGP(10-14)-free vehicle or vehicle containing different OGP(10-14) doses as outlined in FIG. 2. One group of reference baseline controls was left untreated and received neither CFA nor sterile water vehicle with or without OGP(10-14) (FIG. 2). Blood was collected by retroorbital bleeding on days −12, −4, +3, +7, +14, +17, +21 and +24 (FIG. 2C) Differential cell counts were carried out using a Coulter Counter (Sysmex Microcell Counter F-800).

To test the effect of OGP(10-14) on double positive CD34+/Sca-1+ cells in the blood comparatively to that of G-CSF, CFA ablated mice were treated with daily with 10 nmol OGP(10-14) from day −7 until day +7 and blood samples obtained on days +5, +7 and +15 subjected to flow cytometry. G-CSF was administered on days +2 to +8.

For the flow cytometry, groups of three blood samples from the mice were pooled, and mononuclear cells were obtained by gradient centrifugation and were resuspended in PBS at a concentration of 1×106/ml. The cells were then incubated in the presence of specific monoclonal antibodies (final dilution 1:10) for 30 min at 4° C. To detect CD34+ cells, purified rat anti-mouse monoclonal antibody (Pharmingen, RAM34) was used as a first layer. After three washings, the cells were resuspended in PBS and incubated with a FITC polyclonal goat anti-rat (Pharmingen) To detect Sca-1+/CD34+ cells, samples were further washed three times in PBS and incubated with rat anti-mouse Sca-1 (Ly.6A.2) PE from Caltag. Substituting the primary antibody with an irrelevant immunoglobulin performed a specific control. Data acquisition and analysis were assessed by FAC-Scan(tm) (Becton Dickinson) flowcytometer using Lysis II software (FIG. 3).

To evaluate different dosing regimens of OGP(10-14), chemoablated mice were subjected to daily treatment with OGP(10-14), as outlined in FIG. 4. The mice were sacrificed on day +15, the femoral bone marrow was flushed and single cell suspensions (prepared as above) were subjected to ex vivo progenitor cell (colony forming) assays. The OGP(10-14) effect on the formation of CFU-GM, CFU-GEMM and BFU-E was compared to that of G-CSF (FIG. 4).

Progenitor Cell Assays

Bone marrow cells were recovered on day +10 after injection of CFA from all groups. Cells were diluted to 2×106/ml in Iscove's Modified Dulbecco Medium(IMFM) with 2% FBS and added to methylcellulose medium according to the manufacturer's recommendations (MethoCult, StemCell Technologies Inc., Vancouver, Canada). 2×104 cells were plated in each test. Both M3434 (for murine GM-CFU, and GEMM-CFU) and M3334 for (murine BFU-E assay) were used. M3434 was supplemented with recombinant murine interleukine-3 (rmIL-3, 10 ng/ml), recombinant human interleukine-6 (rhIL-6, 10 ng/ml), recombinant murine stem cell factor (rmSCF, 50 ng/ml) and recombinant human erythropoietin (rhEpo, 3 U/ml). InM3434 the only factor included was Epo. Duplicate tests for each mouse were blindly examined after 14 days of incubation according to the protocol procedures.

Results

Total and differential WBC counts carried out on day +3 showd a marked decrease in all CFA-treated groups (FIG. 2). On day 7, there was approximately 2-fold recovery of total WBC counts in the vehicle treated chemoablated, which were still considerably lower than the values recorded in the untreated reference mice. On the other hand, the OGP(10-14) animals showed higher values at all doses tested, with peak counts measured in mice receiving 10 nmol OGP(10-14)/day. The counts in this group approached closely those noted in the untreated reference group (FIG. 2A). Differential cell counts carried out on day 7 also demonstrated an OGP(10-14) induced, dose dependent increase in monocyte and immature cell counts (FIGS. 2B, 2C). Monocyte counts at the maximal dose (10 nmol) were 6-fold higher compared to the normal reference (FIG. 2B); those of immature cells being also significantly higher than the reference (FIG. 2C). The total WBC counts in all animal groups were normal from day 10 and onwards (FIG. 2A). However, the monocyte counts still presented the same trend seen on day 7 with normal levels being reached on day 14 (FIG. 2B). In spite of a decrease in immature cell counts in all but the 0.01 nmol group, the highest values were still obtained in the 10 nmol group. The immature cell counts were normal in all groups from day 14 and onwards (FIG. 2C).

The amount of double positive CD34+/Sca-l+ cells on day +5 was 5-fold higher in the chemoablated animals treated daily with 10 nmol OGP(10-14) than in mice treated with the vehicle alone (FIG. 3). The effect of OGP(10-14) was similar to that of G-CSF. Flow cytometry measurements carried out on days +7 and +15 demonstarted comparable numbers of the CD34+/Sca-l+ cells. However, on day +15, the OGP(10-14) treated mice showed a significantly higher number than the vehicle and G-CSF treated mice (FIG. 3).

The progenitor cell assays showed that OGP(10-14) significantly stimulates CFU-GEMM and BFU-E, but not CFU-GM. The effect of OGP was apparent only in instances where the onset of treatment preceded chemoablation by 7 days (FIG. 4). The absence of an OGP(10-14) effect on CFU-GM is consistent with its non-significant effect on blood granulocyte cell counts. G-CSF had an effect only on the CFU-GM (FIG. 4).

### Example 4

OGP 10-14 Rescues Hematopoietic Bone Marrow Cellularity in Ex-vivo Samples from Patients With Idiopathic Myelofibrosis

Materials and Methods

In order to assess the efficacy of OGP(10-14) in humans, it's hematopoietic activity was studied in ex vivo bone marrow specimens obtain from patients suffering from idiopathic myelofibrosis (IMF). Five IMF patients, one scleroderma patient and two patients with other myelodisplastic syndromes (MDS) were enrolled in the study after signing an informed consent. Diagnosis of IMF was performed on the basis of standard clinical and hematological methods [Barosi, G., et al., Br. J. Haematol. 104:730-737 (1999)]. Bone marrow biopsy showing fibrosis was an essential feature. The diagnosis of IMF was eventually established after excluding other possible causes of fibrosis and the presence of different myeloproliferative disorders. In particular, the diagnosis of chronic myelogenous leukaemia was ruled out by excluding the presence of Ph chromosome and of bcr/abl rearrangement. Three of the five IMF patients had been previously treated by low doses of busulfan administered ten days each month and 1 (g 1,25(OH)2D3/day. The patients' data are summarized in Table.1.

TABLE 1
Clinical data of IMF (A-E) and MDS (F-G) patients

| | Age | Diagnosis | | HB | PLT | WBC | LDHLD | Spleen | |
|---|---|---|---|---|---|---|---|---|---|
| Patient | (years) | date | ECOG | (g/dL) | (X10⁻⁸/L) | (X10⁻⁶/L) | (U/L)* | (cm)** | Treatment |
| | | May 199 [symbol] | 0 | 10.4 | 131 | 15.0 | 740 | 17 | Untreated |
| B | 82 | September 1998 | 2 | 8.5 | 434 | 11.2 | 830 | 20 | Treated |
| C | 78 | March 2000 | 2 | 8.2 | 68 | 1.3 | 664 | 20 | Untreated |
| D | 70 | March 1990 | 3 | 6.8 | 60 | 7.3 | 763 | 30 | Treated |
| E | 79 | June 1995 | 1 | 10.0 | 180 | 4.5 | 666 | 18 | Untreated |
| F | 65 | April 1997 | 0 | 14.0 | 24 | 12.0 | 408 | 30 | Treated |
| G | 65 | February 2000 | 2 | | | | 632 | 11 | Untreated |
| H | 40 | March 2000 | 0 | 15 | 280 | 10 | 300 | 10 | Untreated |

*normal values: 240-480 U/L
**Ecografic measurement

Three mm long bone marrow specimens were obtained from the posterior superior iliac spine by an 8 gauge disposable biopsy needle equipped with a trap to ensure minimal distortion of the specimen (TraoSystem MDThech, USA). The specimens were divided into three,1 cm long portions. One randomly selected portion was used for preliminary morphological assessment. The remaining two fragments were cultured in 35-mm tissue culture dishes and completely covered by 1 ml of Dexter-like medium, in the presence of rhSCF (50 ng/mL), rhGM-CSF (10 ng/mL), rhIL-3 (10 ng/mL), and rhEpo (2 units/mL) with or without 10⁻⁸M OGP(10-14), at 37° C., 5% CO₂-air. Half the medium was changed once, after 7 days, without altering its composition, other than restoring cytokines and OGP(10-14) initial concentration. After additional seven days in culture the bone marrow specimens were histologically processed. Briefly, samples were fixed in modified B5, demineralized in E.D.T.A acid buffer and sections were stained with Giemsa, hematoxylin-eosin or silver impregnation of reticulum. Changes in the bone marrow were assessed semiquantitatively on a I to IV score. A score of IV was used for cell-rich bone marrow specimens comparable to normal ones; score III represented reduced cellularity with reduced nuclear density; score II specimens exhibited spread lacunae; hematopoietic cells in score I specimens were extremely scanty and/or the bone marrow area was vastly substituted by lacunar zones. At least 3 equispaced histological sections per sample were examined using the entire section area. In addition, the cell density was automatically evaluated using a computer assisted Leica microscope equipped with Leica.QWin software, as the ratio between cell counts and bone marrow area. The results for each patient results were expressed as the ratio of mean cell density in OGP(10-14) treated over untreated specimens (T/C ratio).

Results

After 14 days in culture, bone marrow specimens treated with OGP(10-14) appear richer in hematopoietic cells than OGP(10-14)-free specimens from the same patients (FIGS. 5, 6). The semiquantitative score was significantly increased in all IMF patients (p<0.05). No differences were detected between OGP(10-14) treated and untraeted specimens from non IMF patients. The computer assisted evaluation of cellularity showed a T/C ratio>1 in all IMF cases (p<0.01) strongly indicating that cell number was increased in the OGP(10-14)-treated specimens. Moreover, the T/C ratio was statistically significant in, every pair of specimens obtained from individual patients (Table. 2). The T/C ratio showed a very high and significant inverse correlation with the patients' hemoglobin level (FIG. 7). Decreased hemoglobin levels are the most important serological indicator for the severity of IMF. This correlation therefore strongly suggests that the effect of OGP(10-14) is highest in the more severely affected patients.

TABLE 2
Computer assisted evaluation of cell density.

| PATIENT | T/C RATIO | p value |
|---|---|---|
| A | 2.0 | p < 0.05 |
| B | 3.3 | p < 0.01 |
| C | 5.7 | p < 0.003 |
| D | 8.1 | p < 0.0002 |
| E | 1.8 | p < 0.025 |
| F | 1.2 | p = NS |
| G | 0.9 | p = NS |
| H | 1.1 | p = NS |

The ratio between erythroid and myeloid cells was apparently unchanged after culturing with OGP(10-14). However, a semiquantitative assessment suggested a 1.5 to 10-fold decrease in the number of megakaryocytes in specimens obtained from IMF patients. As in the case of overall hematopietic cellularity, such differences were not found in samples obtained from the non-IMF patients.

The invention has been described in detail with respect to preferred embodiments, and it will now be apparent from the foregoing to those skilled in the art, that changes and modifications may be made without departing from the invention in its broader aspects, and the invention, therefore, as defined in the appended claims, is intended to cover all such changes and modifications that fall within the true spirit of the invention.

1. A method for enhancing the mobilization of multilineage hematopoietic stem cells to peripheral blood, comprising the step of administering to a subject in need thereof, an effective amount of an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a pharmaceutical composition comprising the same.

2. The method according to claim 1, wherein said stem cells are multilineage early CD34 positive stem cells.

3. The method according to claim 1, wherein said circulating multilineage stem cells are double positive CD34/Flk2 cells.

4. The method according to claim 1, wherein said oligopeptide is a pentapeptide having the formula: Tyr-Gly-Phe-Gly-Gly, as denoted by the amino acid sequence of SEQ ID NO:1.

5. The method according to claim 1, wherein said oligopeptide is a pentapeptide having the formula: Tyr-Gly-Phe-His-Gly, as denoted by the amino acid sequence of SEQ ID NO:2.

6. The method according to claim 1, wherein said oligopeptide is a tetrapeptide having the formula: Gly-Phe-Gly-Gly, as denoted by the amino acid sequence of SEQ ID NO:3.

7. The method according to claim 1, wherein said oligopeptide is a hexapeptide having the formula: Ac-Met-Tyr-Gly-Phe-Gly-Gly, as denoted by the amino acid sequence of SEQ ID NO:4.

8. A method for enhancement of the number of circulating multilineage early CD34 positive stem cells, comprising the step of administering to a subject in need thereof, an effective amount of an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a pharmaceutical composition comprising the same.

9. The method according to claim 8, wherein said subject in need thereof is a patient receiving irradiation or chemotherapy.

10. The method according to claim 8, wherein said subject suffers from any one of hematological disorders, solid tumors, immunological disorders and aplastic anemia.

11. The method according to claim 10, wherein said hematological disorder is selected from the group consisting of lymphomas, leukemias, Hodgkin's diseases and myeloproliferative disorders.

12. The method according to claim 8, wherein said circulating multilineage stem cells are double positive CD34/Flk2 cells.

13. A method for enhancing the selective proliferation of CD34 positive hematopoietic stem cells in a subject in need thereof, comprising the step of administering to a subject in need thereof, an effective amount of an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a pharmaceutical composition comprising the same.

14. The use according to claim 13, wherein said CD34 positive cells are double positive CD34/Flk2 cells.

15. A method for selectively increasing the number of any one of the BFU-E and GEMM colony forming units (CFU), comprising the step of administering to a subject in need thereof, an effective amount of an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a pharmaceutical composition comprising the same.

16. The method according to claim 13, wherein said subject in need thereof is a patient receiving irradiation or chemotherapy.

17. The method according to claim 16, wherein said subject suffers from any one of hematological disorders, solid tumors, immunological disorders and aplastic anemia.

18. The method according to claim 17, wherein said hematological disorder is selected from the group consisting of lymphomas, leukemias, Hodgkin's diseases and myeloproliferative disorders.

19. A method for enhancing the number of any one of the BFU-E and GEMM colony forming units (CFU) comprising exposing said cells to an effective amount of an oligopeptide comprising the amino acid sequence Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.

20. A method of treating a subject suffering from any one of hematological disorders, solid tumors, immunological disorders and aplastic anemia, comprising administering to said subject a therapeutically effective amount of an oligopeptide having stimulatory activity on hematopoietic cells and comprising the amino acid sequence Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.

21. A method of treating a subject suffering from any one of hematological disorders, solid tumors, immunological disorders and aplastic anemia, comprising administering to said subject a therapeutically effective amount of an oligopeptide having stimulatory activity on hematopoietic cells and comprising the amino acid sequence Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient, in support of the treatment of the subject by bone marrow transplantation.

22. The method according to claim 20, wherein said hematological disorder is any one of a lymphoma, leukemia, Hodgkin's disease and myeloproliferative disorder.

23. A method of treating a subject suffering from a myeloproliferative disorder, comprising administering to said subject a therapeutically effective amount of an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:l, 2, 3 and 4 respectively or of a composition comprising said oligopeptide as an effective ingredient.

24. The method according to claim 23, wherein said myeloproliferative disorder is idiopathic myelofibrosis (IMF).

25. A method for enhancing the proliferation of hematopoietic stem cells comprising exposing said cells to an effective amount of an oligopeptide having stimulatory activity on production of hematopoietic cells and comprising the amino acid sequence Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.

26. A method for enhancing the proliferation of hematopoietic CD34 positive cell stem cells comprising exposing said cells to an effective amount of an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:l, 2, 3 and 4 respectively, or of a composition comprising said oligopeptide as an effective ingredient.

27. The method according to claim 26, wherein said CD34 positive cell is a Flk2 positive cell.

28. A method for the preparation of a peripheral blood stem cell transplant for the treatment of a subject in need thereof comprising the step of administering to a donor an effective amount of an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient, thereby enhancing the mobilization of hematopoietic stem cell to the peripheral blood of said donor and obtaining from said donor a sufficient amount of peripheral blood stem cells.

29. A method for enhancement of engraftment of bone marrow transplants, hemopoietic reconstruction, bone marrow re-population and number of circulating stem cells, which method comprises the step of administering to any one of a cell or of a subject in need thereof, an effective amount of an oligopeptide having stimulatory activity on hematopoietic cells and having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.

30. A method for enhancement of engraftment of bone marrow transplants, hemopoietic reconstruction, bone marrow re-population and number of circulating stem cells in patients receiving chemotherapy or irradiation, which method comprises the step of administering to any one of a cell or of a subject in need thereof, an effective amount of an oligopeptide having stimulatory activity on hematopoietic cells and having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.

31. A method according to claim 29, wherein said oligopeptide increases the number of circulating multilineage stem cells.

32. The method according to claim 31, wherein said multilineage stem cells are the circulating early precursor CD34 positive cells.

33. The method according to claim 32, wherein said multilineage stem cells are the circulating early precursor double positive CD34/Flk2 cells.

34. The method according to claim 29, wherein said oligopeptide enhances the immature cell and monocyte recovery.

35. The method according to claim 29, wherein said oligopeptide selectively increases any one of the BFU-E and GEMM colony forming units (CFU).

36. The method according to claim 29, for supporting bone marrow transplantation by increasing proliferation of stem cells, accelerating the hematological reconstruction upon bone marrow transplantation and increasing the cellularity of bone marrow.

37. A method for reducing acute transplant rejection in a transplanted patient, comprising administering to said patient an effective amount of an oligopeptide having stimulatory activity on hematopoietic cells and comprising the amino acid sequence Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.

38. A method for in vitro and/or ex vivo maintaining and/or expanding stem cell population in a blood sample comprising isolating peripheral blood cells from said blood sample, enriching blood progenitor cells expressing the CD34 antigen, cluttering the enriched blood progenitor cells under suitable conditions, treating said cells with an oligopeptide having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:l, 2, 3 and 4 respectively or with a composition comprising said oligopeptide as an effective ingredient.

39. The method according to claim 38, wherein said cells are in cell culture.

40. The method according to claim 38, wherein said blood sample is mammalian blood sample.

41. The method according to claim 40, wherein said blood sample is a human blood sample.

42. The method according to claim 40, wherein said blood sample originates from a mammal suffering from, or susceptible to decreased blood cell counts.

43. The method according to claim 42, wherein said decreased blood counts are caused by chemotherapy, irradiation therapy, or bone marrow transplantation therapy.

44. The method according to claim 43, wherein said composition further comprises at least one cytokine.

45. The method according to claim 44, wherein said cytokine is selected from the group consisting of TPO (Thrombopoietin), EPO (Erythropoietin), M-CSF (Macrophage-colony stimulating factor), GM-CSF (Granulocyte-macrophage-CSF), G-CSF (Granulocyte CSF), IL-1 (Interleukin-1), IL-2, IL-3, IL-4, IL-5, IL-6, IL-7, IL-8, IL-9, IL-10, IL-12, LIF (Leukemia inhibitory factor) and KL (Kit ligand).

46. A method for re-populating blood cells in a mammal comprising administering to said mammal a therapeutically effective amount of an oligopeptide having stimulatory activity on hematopoietic cells and having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.

47. The method of claim 46, wherein said blood cell is any one of erythroid, myeloid and lymphoid cells.

48. A method for increasing the number of white blood cells, circulating hematopoietic stem cells, and overall bone marrow cellularity comprising administering to a subject a therapeutically effective amount of an oligopeptide having stimulatory activity on hematopoietic cells and having the amino acid sequence of any one of Tyr-Gly-Phe-Gly-Gly, Tyr-Gly-Phe-His-Gly, Gly-Phe-Gly-Gly and Met-Tyr-Gly-Phe-Gly-Gly as denoted by SEQ ID NOs:1, 2, 3 and 4 respectively, or of a composition comprising said oligopeptide as an effective ingredient.


SEQUENCE LISTING (4 sequences)

SEQ ID NO: 1 - protein, 5 aa, Artificial Sequence
  Description of Artificial Sequence synthetic peptide sequence
YGFGG

SEQ ID NO: 2 - protein, 5 aa, Artificial Sequence
  Description of Artificial Sequence synthetic peptide sequence
YGFHG

SEQ ID NO: 3 - protein, 4 aa, Artificial Sequence
  Description of Artificial Sequence synthetic peptide sequence
GFGG

SEQ ID NO: 4 - protein, 6 aa, Artificial Sequence
  Description of Artificial Sequence synthetic peptide sequence
MYGFGG